(19) United States
(12) Patent Application Publication
(10) Pub. No.: US 2024/0294703 A1
(43) Pub. Date: Sep. 5, 2024

(54) AMINE HARDENER WITH HIGH CONTENT IN RENEWABLE CARBON

(71) Applicant: SIKA TECHNOLOGY AG, Baar (CH)

(72) Inventors: Edis KASEMI, Zürich (CH); Ursula STADELMANN, Zürich (CH); Andreas KRAMER, Zürich (CH); Urs BURCKHARDT, Zürich (CH)

(73) Assignee: SIKA TECHNOLOGY AG, Baar (CH)

(21) Appl. No.: 18/563,973

(22) PCT Filed: Jul. 22, 2022
(86) PCT No.: PCT/EP2022/070595

§ 371 Date: Nov. 24, 2023

(30) Foreign Application Priority Data
Jul. 28, 2021 (EP) 21188304.6

(51) Int. Cl. C08G 59/50; C07D 307/52

(52) U.S. Cl. CPC C08G 59/5046; C07D 307/52

(57) ABSTRACT

The use of a hardener that contains at least one amine of formula (I), for curing epoxy resins. The amine of the formula (I) has a high renewable carbon index (RCI) and allows for especially sustainable epoxy resin products. It can be surprisingly easily produced, is low odor, dilutes the epoxy resin especially well and allows rapid and trouble-free hardening with surprising little exothermic effects.

### TECHNICAL FIELD

The invention relates to alkylated amines having a high renewable carbon content and the use thereof as hardeners for epoxy resins.

### PRIOR ART

Amines are used in industry and construction inter alia as hardeners in epoxy resin compositions. Depending on the application, properties demanded include a high reactivity at low exothermicity and/or fast, problem-free curing at ambient temperatures to afford coatings or bodies having uniform surfaces without clouding, staining or cratering due to blushing. The cured bodies or coatings should have a high hardness at low brittleness to resist mechanical stress as well as possible and for optically demanding applications have a high gloss and a low propensity for yellowing. It is crucial for many applications that the epoxy resin composition has a low viscosity in order that it may be applied as quickly and easily as possible, has good levelling and deaerating properties and, in some cases, readily penetrates into the substrates. Since many epoxy resins have a rather high viscosity, an efficient dilution thereof by the amine hardeners is particularly advantageous since this makes it possible to use smaller amounts of the diluent or solvent and/or achieve a higher filler content to establish a suitable viscosity.

There is presently increasing demand for epoxy resin compositions that are sustainable. In particular, they should contain a high content of raw materials from renewable biological sources, i.e. should be biobased to a great extent. There is therefore a need for sustainable amine hardeners. A common measure of the sustainability of chemical raw materials is the Renewable Carbon Index (RCI), which indicates the carbon content from renewable biological sources. It is obtained by dividing the number of carbon atoms derived from renewable sources by the total number of carbon atoms in the raw material.

Hardeners containing amines having a high renewable carbon index are known, for example from U.S. Pat. No. 9,676,898 or WO 2015/124792, which describe bis(aminomethyl)furans and bisfurfurylamines and the use thereof as hardeners for epoxy resins. However, these amines are costly and inconvenient to produce, prone to blushing and their diluting effect on the epoxy resin is in need of improvement.

EP 3,350,245 discloses sustainable hardeners containing alkylated amines having a tetrahydrofuran ring. These hardeners show incomplete curing with reduced final hardness, especially in two-dimensional application and under low-temperature conditions, for example 8° C.

Benzylated amines are also known, for example from EP 2 731 927, EP 3 180 383 or EP 3 344 677. As a hardener for epoxy resins, these amines have a good dilution effect and allow fast, trouble-free curing, even in the case of two-dimensional application and low ambient temperatures. However, biobased production thereof is not possible.

### SUMMARY OF THE INVENTION

It is accordingly an object of the present invention to provide a hardener for epoxy resins which has a high renewable carbon index (RCI), is simple to produce, effects good dilution of the epoxy resin and allows trouble-free curing.

This object is surprisingly achieved with a hardener containing an amine of formula (I) as described in claim 1. The amine of formula (I) is obtained in a simple process from the reductive alkylation of a primary aliphatic amine with furfural based on renewable raw materials. The amine of formula (I) has a high RCI, preferably at least 0.45, in particular at least 0.7.

The hardener containing the amine of formula (I) is capable of surprisingly high dilution. Especially with N-furfuryl-1,2-ethanediamine the epoxy resin is diluted particularly efficiently, even more highly than with the known N-benzyl-1,2-ethanediamine. This is surprising because the amount of N-furfuryl-1,2-ethanediamine required to cure the epoxy resin is lower than for N-benzyl-1,2-ethanediamine due to the lower amine equivalence weight and a lower diluting effect than for N-benzyl-1,2-ethanediamine would be expected due to the oxygen in the furan ring and the resulting possibility of hydrogen bridge formation. The hardener according to the invention is very low-odor which is a further great advantage for many applications. It enables fast and trouble-free curing to a high final hardness. What is particularly surprising is the low exothermicity during curing which is markedly lower than when using N-benzyl-1,2-ethanediamine, the processing times and curing rates with N-furfuryl-1,2-ethandiamine being only insubstantially longer/slower than with N-benzyl-1,2-ethanediamine. The low exothermicity allows use in epoxy resin products employed in thick layers such as shaped bodies, potting compounds or matrix resins for composites, without occurrence of blisters, discoloration or other inhomogeneities owing to high evolution of heat. In the case of two-dimensional use the hardener makes it possible to achieve epoxy resin coatings curable at ambient temperatures with attractive, glossy surfaces and only very low propensity for blushing effects. It is also particularly surprising that N-furfuryl-1,2-ethanediamine may also be used in the form of a little-purified, particularly inexpensively producible reaction product without appreciable adverse effects during curing of the epoxy resins.

Further aspects of the invention are the subject of further independent claims. Particularly preferred embodiments of the invention are the subject of the dependent claims.

### Ways of Executing the Invention

The invention provides for use of a hardener containing at least one amine of formula (I) for curing of epoxy resins,

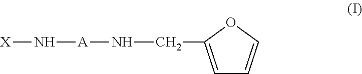

wherein A represents a linear alkylene radical having 2 to 10 carbon atoms and X represents H or furfuryl.

“RCI” is the “Renewable Carbon Index” of a substance or a mixture of substances, wherein the RCI is the ratio of the number of carbon atoms from biobased sources to the total number of carbon atoms of the substance or the mixture of substances. A “primary amino group” refers to an amino group that is attached to a single organic radical and bears two hydrogen atoms; a “secondary amino group” refers to an amino group that is attached to two organic radicals, which may also together be part of a ring, and bears one hydrogen atom; and a “tertiary amino group” refers to an amino group that is attached to three organic radicals, two or three of which may also be part of one or more rings, and does not bear any hydrogen atoms.

“Amine hydrogen” refers to the hydrogen atoms of primary and secondary amine groups.

“Amine hydrogen equivalent weight” refers to the mass of an amine or an amine-containing composition that contains one molar equivalent of amine hydrogen. It is expressed in units of “g/eq”.

The “epoxy equivalent weight” refers to the mass of an epoxy group-containing compound or composition that contains one molar equivalent of epoxy groups. It is expressed in units of “g/eq”.

Substance names beginning with “poly”, such as polyamine or polyepoxide, refer to substances that formally contain two or more of the functional groups that occur in their name per molecule.

A “diluent” refers to a substance that is soluble in an epoxy resin and lowers its viscosity and that is not chemically incorporated into the epoxy resin polymer during the curing process.

“Molecular weight” refers to the molar mass (in grams per mole) of a molecule.

“Average molecular weight” refers to the number-average Mₙ of a polydisperse mixture of oligomeric or polymeric molecules, which is normally determined by gel-permeation chromatography (GPC) against polystyrene as standard.

“Pot life” refers to the maximum period of time from the mixing of the components to the application of an epoxy resin composition in which the mixed composition is in a sufficiently free-flowing state and has good ability to wet the substrate surfaces.

The “gel time” is the time interval from the mixing of the components of an epoxy resin composition to gelation thereof.

“Room temperature” refers to a temperature of 23° C.

All industry standards and norms mentioned in this document refer to the versions valid at the date of first filing, unless otherwise stated.

Percent by weight (% by weight) values refer to the mass fractions of a constituent in a composition based on the overall composition, unless otherwise stated. The terms “mass” and “weight” are used synonymously in the present document.

A preferably represents 1,2-ethylene, 1,3-propylene, 1,4-butylene, 1,5-pentylene, 1,6-hexylene, 1,7-heptylene, 1,8-octylene, 1,9-nonylene or 1,10-decylene.

A is particularly preferably selected from the group consisting of 1,2-ethylene, 1,3-propylene, 1,4-butylene, 1,5-pentylene and 1,6-hexylene. These amines are obtainable particularly simply and with high RCI and have particularly good compatibility with epoxy resins.

A most preferably represents 1,2-ethylene. Such an amine of formula (I) makes it possible to achieve epoxy resin compositions with particularly fast and trouble-free curing and has a particularly high RCI, even in the event that the carbon atoms from the radical A do not originate from a biobased source.

It is preferable when X represents H. Such an amine of formula (I) dilutes the epoxy resin particularly well and makes it possible to achieve particularly rapid curing and particularly high final hardnesses.

It is preferable when the amine of formula (I) is selected from the group consisting of N-furfuryl-1,2-ethanediamine, N,N′-difurfuryl-1,2-ethanediamine, N-furfuryl-1,3-propanediamine, N,N′-difurfuryl-1,3-propanediamine, N-furfuryl-1,4-butanediamine, N,N′-difurfuryl-1,4-butanediamine, N-furfuryl-1,5-pentanediamine, N,N′-difurfuryl-1,5-pentanediamine, N-furfuryl-1,6-hexanediamine and N,N′-difurfuryl-1,6-hexanediamine.

Preferred among these are N-furfuryl-1,2-ethanediamine or N,N′-difurfuryl-1,2-ethanediamine. N-Furfuryl-1,2-ethanediamine is particularly preferred.

In a preferred embodiment of the invention the amine of formula (I) is employed as a mixture of amine of formula (I) where X═H and amine of formula (I) where X═furfuryl in a weight ratio in the range from 50/50 to 98/2, in particular 60/40 to 95/5. Such a mixture is particularly inexpensively producible and allows fast and trouble-free curing of the epoxy resin.

The amine of formula (I) is preferably produced by reductive alkylation of at least one amine of formula H₂N-A-NH₂ with furfural and hydrogen.

Furfural is preferably based on renewable raw materials and has an RCI of 1. This allows amines of formula (I) having a high RCI.

Commercially available furfural typically originates from a biobased source. On a large industrial scale furfural is obtained for example from hemicellulose from vegetable materials, in particular by the action of sulfuric acid on the C5 sugars present therein in a dehydration, or in pulp production according to the magnesium bisulfite process, where liberated furfural can be extracted from the black liquor.

Preferred amines of formula H₂N-A-NH₂ are 1,2-ethanediamine, 1,3-propanediamine, 1,4-butanediamine, 1,5-pentanediamine or 1,6-hexanediamine, in particular 1,2-ethanediamine.

In a preferred embodiment of the invention the carbon atoms of the amine of formula H₂N-A-NH₂ also originate from a renewable source. This makes it possible to achieve very particularly sustainable amines of formula (I), in particular having an RCI of 1. It is preferable when the amine of formula (I) has an RCI of at least 0.45, preferably at least 0.6, in particular at least 0.7, most preferably of 1.

The reductive alkylation is preferably performed in the presence of a suitable catalyst. Preferred catalysts are palladium on charcoal (Pd/C), platinum on charcoal (Pt/C), Adams' catalyst or Raney nickel, especially palladium on charcoal or Raney nickel.

The reductive alkylation is preferably performed in a pressure apparatus at a hydrogen pressure of 5 to 120 bar, in particular 10 to 100 bar. This may be effected in a batchwise process or preferably in a continuous process.

The reductive alkylation is preferably performed at a temperature in the range from 40° C. to 120° C., especially 60° C. to 100° C.

Depending on the stoichiometry between the amine of formula H₂N-A-NH₂ and furfural the obtained reaction mixture contains different proportions of monoalkylated amine of formula H₂N-A-NH₂, i.e. amine of formula (I) where X═H, and dialkylated amine of formula H₂N-A-NH₂, i.e. amine of formula (I) where X═furfuryl.

In the event that an amine of formula (I) where X═furfuryl is to be produced the molar ratio of the amine of formula H₂N-A-NH₂ to furfural is preferably in the range from 0.4 to 0.7, in particular 0.5. A thus-obtained reaction mixture contains a particularly high content of amine of formula (I) where X═furfuryl.

In the event that an amine of formula (I) where X═H is to be produced the molar ratio of the amine of formula H₂N-A-NH₂ to furfural is preferably in the range from 1 to 10, in particular 1 to 5. A thus-obtained reaction mixture contains a high content of amine of formula (I) where X═H.

Excess amine of formula H₂N-A-NH₂ is preferably removed from the reaction mixture after the reaction, in particular by distillation together with the released water.

The reaction mixture may be further purified, in particular by distillation/fractionation. The amine of formula (I) may be freed of the byproducts and/or the amine of formula (I) where X═H may be separated from the amine of formula (I) where X═furfuryl.

It is preferable when the amine of formula (I) is employed in the form of a reaction product obtained from the reductive alkylation of at least one amine of formula H₂N-A-NH₂ with furfural and hydrogen and subsequent removal of unreacted amine of formula H₂N-A-NH₂.

The reaction product is preferably not further purified, in particular a distillation/fractionation of the amines of formula (I) is eschewed.

Such a reaction product is producible particularly inexpensively. It contains a low content of amine of formula H₂N-A-NH₂, preferably less than 2% by weight, particularly preferably less than 1% by weight, especially less than 0.5% by weight, of amine of formula H₂N-A-NH₂ based on the total reaction product.

The reaction product may contain byproducts from the reductive alkylation, in particular amines having di- or tri-alkylated nitrogen atoms, and amines having a hydrogenated furan ring. These proportions are preferably low.

It is preferable when the reaction product has a content of amines having di- or tri-alkylated nitrogen atoms, in particular of formulae

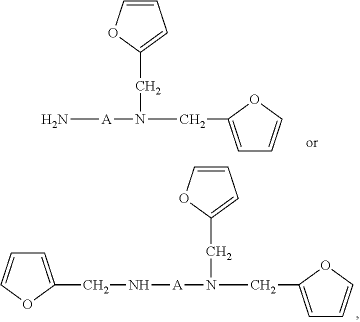

of altogether less than 10% by weight, particularly preferably less than 5% by weight, in particular less than 2% by weight, based on the total reaction product.

It is preferable when the reaction product has a content of amines having a hydrogenated furan ring, in particular of formulae

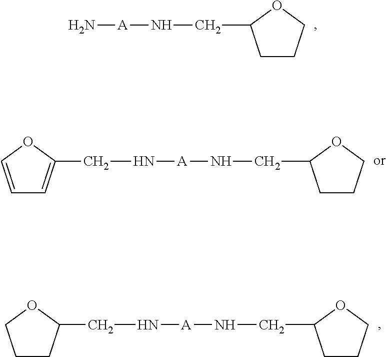

of altogether less than 20% by weight, in particular less than 15% by weight, based on the total reaction product.

In a particularly preferred embodiment of the invention the amine of formula (I) is obtained in the form of a reaction product from the reductive alkylation of at least one amine of formula H₂N-A-NH₂ with furfural and hydrogen and subsequent removal of unreacted amine of formula H₂N-A-NH₂, wherein the molar ratio of the amine of formula H₂N-A-NH₂ to furfural is in the range from 1 to 2, preferably 1 to 1.5. It is preferable when A represents 1,2-ethylene.

The content of amine of formula H₂N-A-NH₂ in this reaction product is preferably at most 1% by weight, particularly preferably at most 0.5% by weight, in particular at most 0.2% by weight, based on the total reaction product.

Such a reaction product contains a surprisingly high content of amine of formula (I) where X═H and surprisingly little amine of formula (I) where X═furfuryl and has a surprisingly high reactivity towards the epoxy resin which is hardly inferior to that of the largely pure amine of formula (I) where X═H. This could not have been expected from the prior art. A corresponding reaction of 1,2-ethanediamine with benzaldehyde instead of furfural has a massively higher content of N,N′-dialkylated 1,2-ethanediamine at a corresponding stoichiometry.

It is preferable when the weight ratio between the amine of formula (I) where X═H and the amine of formula (I) where X═furfuryl in the reaction product is in the range from 50/50 to 98/2, preferably 60/40 to 95/5, based on the reaction product.

The invention thus further provides the reaction product obtained from the reductive alkylation of an amine of formula H₂N-A-NH₂ with furfural and hydrogen in a molar ratio of the amine of formula H₂N-A-NH₂ to furfural in the range from 1 to 2, preferably 1 to 1.5, and subsequent removal of amine of formula H₂N-A-NH₂ to a content of at most 1% by weight, preferably at most 0.5% by weight, in particular at most 0.2% by weight, based on the reaction product, wherein A represents a linear alkylene radical having 2 to 10 carbon atoms, in particular 1,2-ethylene.

It is preferable when A represents 1,2-ethylene and the reaction product contains 50% to 80% by weight of N-furfuryl-1,2-ethanediamine, 5% to 50% by weight, in particular 5% to 40% by weight, of N,N′-difurfuryl-1,2-ethanediamine,
0% to 20% by weight, in particular 2% to 15% by weight, of N-tetrahydrofurfuryl-1,2-ethanediamine,
less than 1% by weight, preferably less than 0.5% by weight, in particular less than 0.2% by weight, of 1,2-ethanediamine and optionally further constituents, in particular further byproducts from the reductive alkylation, based on the reaction product.

Such a reaction product is easily and inexpensively producible and without further purification very well suited as a constituent of a hardener for hardening of epoxy resins, wherein said product has a high reactivity towards the epoxy resin which is hardly inferior to that of substantially pure N-furfuryl-1,2-ethanediamine.

It is preferable to employ an amine of formula (I) where X═H as a mixture with an amine of formula

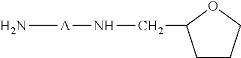

in a weight ratio between the amine of formula (I) where X═H and the amine of formula

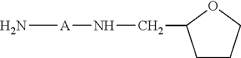

in the range from 70/30 to 99/1, preferably 80/20 to 98/2, wherein A is as specified above.

Such an amine mixture is simple to produce and allows surprisingly fast and trouble-free curing of the epoxy resin.

The invention thus further provides an amine mixture containing at least one amine of formula

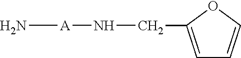

and at least one amine of formula

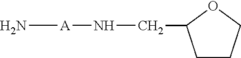

in a weight ratio in the range from 70/30 to 99/1, preferably 80/20 to 98/2, wherein A represents a linear alkylene radical having 2 to 10 carbon atoms, in particular 1,2-ethylene.

In a preferred embodiment of the invention the amine of formula (I) is used partially or completely in the form of an amine-functional adduct with at least one epoxy resin or monoepoxide in a stoichiometric ratio of at least 1 mol of amine of formula (I) to 1 mol equivalent of epoxy groups.

Such an adduct is in the form of a mixture of adducted molecules having at least two, typically having 3 or 4, amine hydrogens derived from the amine of formula (I) and free, non-adducted amine of formula (I). It allows particularly fast curing at moderate viscosity, especially also at low temperatures of 8° C.

The epoxy resin preferably has an average epoxy equivalent weight in the range from 150 to 500 g/eq, preferably 156 to 250 g/eq.

Preference is given to aromatic epoxy resins having an average functionality in the range from 2 to 4, in particular a bisphenol A, F or A/F diglycidyl ether or a novolac epoxy resin. These adducts allow particularly fast curing and high glass transition temperatures.

Also preferred are epoxy resins having polyoxypropylene and/or polyoxyethylene units. These are especially diglycidyl ethers of polypropylene glycols or reaction products of bisphenol A, F or A/F diglycidyl ethers with polypropylene glycols or polyethylene glycols. Such adducts are particularly suitable as a constituent of water-based hardeners for epoxy resins.

Particular preference is given to aromatic diepoxides, in particular a bisphenol A, F or A/F diglycidyl ether.

Very particular preference is given to a bisphenol A diglycidyl ether having an RCI of 0.28 from the reaction of bisphenol A with biobased epichlorohydrin. This makes it possible to obtain particularly sustainable adducts.

It is preferable when the adducting is carried out in a stoichiometric ratio in the range from 1 to 10, preferably 1.2 to 5, in particular 1.4 to 3, mol of amine of formula (I) per molar equivalent of epoxy groups.

The hardener preferably contains at least one further constituent selected from further amines which do not conform to formula (I), accelerators and diluents, in particular at least one further amine which does not conform to formula (I).

It is preferable when the hardener contains at least one further amine which does not conform to formula (I) and which is not a byproduct from the production of the amine of formula (I).

Preferred further amines which do not conform to formula (I) are amines having aliphatic amino groups and at least three amine hydrogens, in particular N-benzyl-1,2-ethanediamine, N-benzyl-1,2-propanediamine, N-benzyl-1,3-bis(aminomethyl)benzene, N-(2-ethylhexyl)-1,3-bis(aminomethyl)benzene, 2,2-dimethyl-1,3-propanediamine, 1,3-pentanediamine (DAMP), 1,5-pentanediamine, 1,5-diamino-2-methylpentane (MPMD), 2-butyl-2-ethyl-1,5-pentanediamine (C11-neodiamine), 1,6-hexanediamine, 2,5-dimethyl-1,6-hexanediamine, 2,2(4),4-trimethyl-1,6-hexanediamine (TMD), 1,7-heptanediamine, 1,8-octanediamine, 1,9-nonanediamine, 1,10-decanediamine, 1,11-undecanediamine, 1,12-dodecanediamine, 1,2-, 1,3- or 1,4-diaminocyclohexane, 1,3-bis(aminomethyl)cyclohexane, 1,4-bis(aminomethyl)cyclohexane, bis(4-aminocyclohexyl)methane, bis(4-amino-3-methylcyclohexyl)methane, bis(4-amino-3-ethylcyclohexyl)methane, bis(4-amino-3,5-dimethylcyclohexyl)methane, bis(4-amino-3-ethyl-5-methylcyclohexyl)methane, 1-amino-3-aminomethyl-3,5,5-trimethylcyclohexane (IPDA), 2(4)-methyl-1,3-diaminocyclohexane, 2,5(2,6)-bis(aminomethyl)bicyclo[2.2.1]heptane (NBDA), 3(4),8(9)-bis(aminomethyl)tricyclo[5.2.1.0^{2,6}]decane, 1,4-diamino-2,2,6-trimethylcyclohexane (TMCDA), 1,8-menthanediamine, 3,9-bis(3-aminopropyl)-2,4,8,10-tetraoxaspiro[5.5]undecane, 1,3-bis(aminomethyl)benzene (MXDA), 1,4-bis(aminomethyl)benzene, bis(2-aminoethyl) ether, 3,6-dioxaoctane-1,8-diamine, 4,7-dioxadecane-1,10-diamine, 4,7-dioxadecane-2,9-diamine, 4,9-dioxadodecane-1,12-diamine, 5,8-dioxadodecane-3,10-diamine, 4,7,10-trioxatridecane-1,13-diamine or higher oligomers of these diamines, bis(3-aminopropyl)polytetrahydrofurans or other polytetrahydrofurandiamines, polyoxyalkylenedi- or -triamines, in particular polyoxypropylenediamines or polyoxypropylenetriamines such as Jeffamine® D-230, Jeffamine® D-400 or Jeffamine® T-403 (all from Huntsman), furan-based amines such as 2,5-bis(aminomethyl)furan, 2,5-bis(aminomethyl)tetrahydrofuran, bis(5-aminomethylfuran-2-yl)methane, bis(5-aminomethyltetrahydrofuran-2-yl)methane, 2,2-bis(5-aminomethylfuran-2-yl)propane or 2,2-bis(5-aminomethyltetrahydrofuran-2-yl)propane, or diethylenetriamine (DETA), triethylenetetramine (TETA), tetraethylenepentamine (TEPA), pentaethylenehexamine (PEHA), dipropylenetriamine (DPTA), N-(2-aminoethyl)-1,3-propanediamine (N3-amine), N,N′-bis(3-aminopropyl)ethylenediamine (N4-amine), N,N′-bis(3-aminopropyl)-1,4-diaminobutane, N5-(3-aminopropyl)-2-methyl-1,5-pentanediamine, N3-(3-aminopentyl)-1,3-pentanediamine, N5-(3-amino-1-ethylpropyl)-2-methyl-1,5-pentanediamine, N,N′-bis(3-amino-1-ethylpropyl)-2-methyl-1,5-pentanediamine, 3-(2-aminoethyl)aminopropylamine, bis(hexamethylene)triamine (BHMT), N-aminoethylpiperazine, 3-dimethylaminopropylamine (DMAPA), 3-(3-(dimethylamino)propylamino)propylamine (DMAPAPA), amine-functional adducts of the recited amines with epoxides, phenalkamines, which are reaction products of cardanol with aldehydes, in particular formaldehyde, and polyamines, or a mixture of two or more of these amines.

The hardener preferably contains at least one amine selected from the group consisting of N-benzyl-1,2-ethanediamine, N,N′-dibenzyl-1,2-ethanediamine, MPMD, TMD, 1,2-diaminocyclohexane, 1,3-bis(aminomethyl)cyclohexane, 1,4-bis(aminomethyl)cyclohexane, bis(4-aminocyclohexyl)methane, IPDA, 2(4)-methyl-1,3-diaminocyclohexane, MXDA, DETA, TETA, TEPA, N3-amine, N4-amine, DPTA, BHMT, polyoxypropylenediamines having an average molecular weight Mₙ in the range from 200 to 500 g/mol, polyoxypropylenetriamines having an average molecular weight Mₙ in the range from 300 to 500 g/mol, 2,5-bis(aminomethyl)furan, 2,5-bis(aminomethyl)tetrahydrofuran, bis(5-aminomethylfuran-2-yl)methane, bis(5-aminomethyltetrahydrofuran-2-yl)methane, 2,2-bis(5-aminomethylfuran-2-yl)propane, 2,2-bis(5-aminomethyltetrahydrofuran-2-yl)propane and phenalkamines.

Preference among these is given to 1,3-bis(aminomethyl)cyclohexane or 1,4-bis(aminomethyl)cyclohexane, especially 1,3-bis(aminomethyl)cyclohexane. This permits particularly rapid curing.

Further preferred among these is IPDA. This achieves particularly high glass transition temperatures which allows particularly good robustness towards high usage temperatures. It is particularly preferable to employ IPDA having a high RCI from biobased acetone which makes it possible to achieve particularly sustainable hardeners.

Further preferred among these is MXDA. This achieves high curing rates and particularly high strengths.

Further preferred among these is N-benzyl-1,2-ethanediamine. Such a hardener allows particularly low-viscosity epoxy resin products with particularly attractive surfaces.

Further preferred among these are 2,5-bis(aminomethyl)furan, 2,5-bis(aminomethyl)tetrahydrofuran, bis(5-aminomethylfuran-2-yl)methane, bis(5-aminomethyltetrahydrofuran-2-yl)methane, 2,2-bis(5-aminomethylfuran-2-yl)propane or 2,2-bis(5-aminomethyltetrahydrofuran-2-yl)propane, in particular 2,5-bis(aminomethyl)furan. This makes it possible to achieve particularly sustainable hardeners.

The hardener may in particular contain more than one further amine which does not conform to formula (I).

The hardener particularly preferably contains as a further amine which does not conform to formula (I) at least one amine having an RCI of 1, in particular selected from 2,5-bis(aminomethyl)furan, 2,5-bis(aminomethyl)tetrahydrofuran, bis(5-aminomethylfuran-2-yl)methane, bis(5-aminomethyltetrahydrofuran-2-yl)methane, 2,2-bis(5-aminomethylfuran-2-yl)propane and 2,2-bis(5-aminomethyltetrahydrofuran-2-yl)propane.

It is preferable when the hardener contains an amount of further amines which do not conform to formula (I) such that 5% to 95%, preferably 10% to 80%, in particular 15% to 60%, of all amine hydrogens present originate from amines of formula (I). In the event that the amine of formula (I) is present in the form of an adduct with an epoxy resin the amine hydrogens of such adducts are likewise counted as amine hydrogens of the amine of formula (I).

Suitable accelerators are especially acids or compounds hydrolyzable to acids, especially organic carboxylic acids such as acetic acid, benzoic acid, salicylic acid, 2-nitrobenzoic acid, lactic acid, organic sulfonic acids such as methanesulfonic acid, p-toluenesulfonic acid or 4-dodecylbenzenesulfonic acid, sulfonic esters, other organic or inorganic acids, such as phosphoric acid in particular, or mixtures of the abovementioned acids and acid esters; nitrates such as calcium nitrate in particular; tertiary amines such as, in particular, 1,4-diazabicyclo[2.2.2]octane, benzyldimethylamine, α-methylbenzyldimethylamine, triethanolamine, dimethylaminopropylamine, imidazoles such as, in particular, N-methylimidazole, N-vinylimidazole or 1,2-dimethylimidazole, salts of such tertiary amines, quaternary ammonium salts, such as benzyltrimethylammonium chloride in particular, amidines, such as 1,8-diazabicyclo[5.4.0]undec-7-ene in particular, guanidines, such as 1,1,3,3-tetramethylguanidine in particular, phenols, especially bisphenols, phenolic resins or Mannich bases such as, in particular, 2-(dimethylaminomethyl)phenol, 2,4,6-tris(dimethylaminomethyl)phenol or polymers produced from phenol, formaldehyde and N,N-dimethylpropane-1,3-diamine, phosphites such as, in particular, di- or triphenyl phosphites, or compounds having mercapto groups.

Preference is given to acids, nitrates, tertiary amines or Mannich bases, especially salicylic acid, calcium nitrate or 2,4,6-tris(dimethylaminomethyl)phenol, or a combination of these accelerators.

Suitable diluents are especially n-propanol, isopropanol, n-butanol, isobutanol, tert-butanol, 1-pentanol, 2-pentanol, 3-pentanol, 2-methyl-1-butanol, 3-methyl-1-butanol, 3-methyl-2-butanol, n-hexanol, 2-ethylhexanol, xylene, 2-methoxyethanol, dimethoxyethanol, 2-ethoxyethanol, 2-propoxyethanol, 2-isopropoxyethanol, 2-butoxyethanol, 2-phenoxyethanol, 2-benzyloxyethanol, benzyl alcohol, ethylene glycol, ethylene glycol dimethyl ether, ethylene glycol diethyl ether, ethylene glycol dibutyl ether, ethylene glycol diphenyl ether, diethylene glycol, diethylene glycol monomethyl ether, diethylene glycol monoethyl ether, diethylene glycol mono-n-butyl ether, diethylene glycol dimethyl ether, diethylene glycol diethyl ether, diethylene glycol di-n-butyl ether, propylene glycol butyl ether, propylene glycol phenyl ether, dipropylene glycol, dipropylene glycol monomethyl ether, dipropylene glycol dimethyl ether, dipropylene glycol di-n-butyl ether, 2,2,4-trimethylpentane-1,3-diol monoisobutyrate, diphenylmethane, diisopropylnaphthalene, mineral oil fractions, for example Solvesso® grades (Exxon), alkylphenols such as tert-butylphenol, nonylphenol, dodecylphenol, cardanol, styrenated phenol, bisphenols, aromatic hydrocarbon resins, especially types containing phenol groups, alkoxylated phenol, especially ethoxylated or propoxylated phenol, especially 2-phenoxyethanol, adipates, sebacates, phthalates, benzoates, organic phosphoric or sulfonic esters or sulfonamides.

Preference among these is given to diluents having a boiling point of more than 200° C., especially benzyl alcohol, styrenated phenol, ethoxylated phenol, aromatic hydrocarbon resins containing phenol groups, such as in particular the Novares® grades LS 500, LX 200, LA 300 or LA 700 (from Ruitgers), diisopropylnaphthalene or cardanol, especially benzyl alcohol.

Phenol-containing diluents are also effective as accelerators.

Preference among these is given also to aromatic diluents having a particularly high diluting effect, especially xylene.

Particular preference among these is given to diluents having an RCI of 1, in particular cardanol. These make it possible to achieve a particularly sustainable hardener.

The hardener preferably contains only a small content of diluents, in particular 0% to 50% by weight, preferably 0% to 30% by weight, of diluents based on the overall hardener.

The hardener preferably contains 1% to 99% by weight, more preferably 5% to 90% by weight, more preferably 10% to 80% by weight, particularly preferably 15% to 70% by weight, of amines of formula (I) based on the overall hardener.

The hardener may be water-based and contain water in the range from 15% to 90% by weight, preferably 20% to 80% by weight.

The hardener is preferably not water-based. It preferably contains less than 15% by weight, especially less than 10% by weight, of water, based on the overall hardener. Such a hardener is particularly suitable for nonaqueous epoxy resin products.

The hardener may contain further constituents, especially:
further adducts, especially adducts of MPMD or 1,2-ethanediamine or 1,2-propanediamine with cresyl glycidyl ether or aromatic epoxy resins, in which unreacted MPMD, 1,2-ethanediamine or 1,2-propanediamine has been removed by distillation after the reaction,
monoamines such as, in particular, benzylamine or furfurylamine,
polyamidoamines, especially reaction products of a mono- or polybasic carboxylic acid, or the ester or anhydride thereof, especially a dimer fatty acid, with a polyamine used in stoichiometric excess, especially DETA or TETA,
Mannich bases,
aromatic polyamines such as in particular 4,4′-, 2,4′ and/or 2,2′-diaminodiphenylmethane, 2,4(6)-toluenediamine, 3,5-dimethylthio-2,4(6)-toluenediamine or 3,5-diethyl-2,4(6)-tolylenediamine,
compounds having mercapto groups, especially liquid mercaptan-terminated polysulfide polymers, mercaptan-terminated polyoxyalkylene ethers, mercaptan-terminated polyoxyalkylene derivatives, polyesters of thiocarboxylic acids, 2,4,6-trimercapto-1,3,5-triazine, triethylene glycol dimercaptan or ethanedithiol,
surface-active additives, especially defoamers, deaerating agents, wetting agents, dispersants or levelling agents, or
stabilizers, especially stabilizers against oxidation, heat, light or UV radiation.

The invention further provides an epoxy resin composition comprising
a resin component comprising at least one epoxy resin and
a hardener component comprising the hardener containing at least one amine of formula (I) as described hereinabove.

A suitable epoxy resin is obtained in a known manner, especially from the reaction of epichlorohydrin with polyols, polyphenols or amines.

Suitable epoxy resins are especially aromatic epoxy resins, especially the glycidyl ethers of:
bisphenol A, bisphenol F or bisphenol A/F, where A stands for acetone and F for formaldehyde used as reactants in the production of these bisphenols. In the case of bisphenol F, positional isomers may also be present, more particularly ones derived from 2,4′- or 2,2′-hydroxyphenylmethane.
dihydroxybenzene derivatives such as resorcinol, hydroquinone or catechol;
further bisphenols or polyphenols such as bis(4-hydroxy-3-methylphenyl)methane, 2,2-bis(4-hydroxy-3-methylphenyl)propane (bisphenol C), bis(3,5-dimethyl-4-hydroxyphenyl)methane, 2,2-bis(3,5-dimethyl-4-hydroxyphenyl)propane, 2,2-bis(3,5-dibromo-4-hydroxyphenyl)propane, 2,2-bis(4-hydroxy-3-tert-butylphenyl)propane, 2,2-bis(4-hydroxyphenyl)butane (bisphenol B), 3,3-bis(4-hydroxyphenyl)pentane, 3,4-bis(4-hydroxyphenyl)hexane, 4,4-bis(4-hydroxyphenyl)heptane, 2,4-bis(4-hydroxyphenyl)-2-methylbutane, 2,4-bis(3,5-dimethyl-4-hydroxyphenyl)-2-methylbutane, 1,1-bis(4-hydroxyphenyl)cyclohexane (bisphenol Z), 1,1-bis(4-hydroxyphenyl)-3,3,5-trimethylcyclohexane (bisphenol TMC), 1,1-bis(4-hydroxyphenyl)-1-phenylethane, 1,4-bis[2-(4-hydroxyphenyl)-2-propyl]benzene (bisphenol P), 1,3-bis[2-(4-hydroxyphenyl)-2-propyl]benzene (bisphenol M), 4,4′-dihydroxydiphenyl (DOD), 4,4′-dihydroxybenzophenone, bis(2-hydroxynaphth-1-yl)methane, bis(4-hydroxynaphth-1-yl)methane, 1.5-dihydroxynaphthalene, tris(4-hydroxyphenyl)methane, 1,1,2,2-tetrakis(4-hydroxyphenyl)ethane, bis(4-hydroxyphenyl) ether or bis(4-hydroxyphenyl) sulfone;
novolacs, which are especially condensation products of phenol or cresols with formaldehyde or paraformaldehyde or acetaldehyde or crotonaldehyde or isobutyraldehyde or 2-ethylhexanal or benzaldehyde or furfural;
aromatic amines such as aniline, toluidine, 4-aminophenol, 4,4′-methylenediphenyldiamine, 4,4′-methylenediphenyldi(N-methyl)amine, 4,4′-[1,4-phenylenebis(1-methylethylidene)]bisaniline (bisaniline P) or 4,4′-[1,3-phenylenebis(1-methylethylidene)]bisaniline (bisaniline M).

Further suitable epoxy resins are aliphatic or cycloaliphatic polyepoxides, especially
glycidyl ethers of saturated or unsaturated, branched or unbranched, cyclic or open-chain di-, tri- or tetrafunctional C₂ to C₃₀ alcohols, especially ethylene glycol, propylene glycol, butylene glycol, hexanediol, octanediol, polypropylene glycols, dimethylolcyclohexane, neopentyl glycol, dibromoneopentyl glycol, castor oil, trimethylolpropane, trimethylolethane, pentaerythritol, sorbitol or glycerol, or alkoxylated glycerol or alkoxylated trimethylolpropane;
a hydrogenated bisphenol A, F or A/F liquid resin or the glycidylation products of hydrogenated bisphenol A, F or A/F;
an N-glycidyl derivative of amides or heterocyclic nitrogen bases, such as triglycidyl cyanurate or triglycidyl isocyanurate, or reaction products of epichlorohydrin with hydantoin.

Further suitable epoxy resins are epoxy resins having a high RCI, especially those from the reaction of biobased hydroxy-functional raw materials with epichlorohydrin. Particular preference is given to vanillin-based epoxy resins such as especially diglycidyl ethers of vanillin alcohol and glycerol-based epoxy resins such as especially triglycidyl ethers of biobased glycerol.

The epoxy resin is preferably a liquid resin or a mixture containing two or more liquid epoxy resins.

“Liquid epoxy resin” refers to an industrial polyepoxide having a glass transition temperature below 25° C.

The resin component optionally additionally contains proportions of solid epoxy resin.

The epoxy resin is especially a liquid resin based on a bisphenol or novolac, especially having an average epoxy equivalent weight in the range from 156 to 210 g/eq.

Particularly suitable are a bisphenol A diglycidyl ether and/or bisphenol F diglycidyl ether, such as are commercially available for example from Olin, Huntsman or Momentive. These liquid resins have low viscosity for epoxy resins and permit rapid curing and high hardnesses. They may contain proportions of solid bisphenol A resin or novolac epoxy resins.

Very particular preference is given to a bisphenol A diglycidyl ether having an RCI of 0.28 from the reaction of bisphenol A with biobased epichlorohydrin. This makes it possible to achieve a particularly sustainable epoxy resin composition.

Also particularly suitable are phenol-formaldehyde novolac glycidyl ethers, especially having an average functionality in the range from 2.3 to 4, preferably 2.5 to 3. These may contain proportions of other epoxy resins, in particular bisphenol A diglycidyl ether or bisphenol F diglycidyl ether.

Also particularly suitable are diglycidyl ethers of vanillin alcohol or triglycidyl ethers of glycerol, in particular diglycidyl ethers of vanillin alcohol.

The resin component may comprise a reactive diluent.

Preferred reactive diluents are reactive diluents containing epoxy groups, especially butanediol diglycidyl ether, hexanediol diglycidyl ether, trimethylolpropane di- or triglycidyl ether, phenyl glycidyl ether, cresyl glycidyl ether, guaiacol glycidyl ether, 4-methoxyphenyl glycidyl ether, p-n-butylphenyl glycidyl ether, p-tert-butylphenyl glycidyl ether, 4-nonylphenyl glycidyl ether, 4-dodecylphenyl glycidyl ether, cardanol glycidyl ether, benzyl glycidyl ether, allyl glycidyl ether, butyl glycidyl ether, hexyl glycidyl ether, 2-ethylhexyl glycidyl ether, or glycidyl ethers of natural alcohols, such as, in particular, C₈ to C₁₀ or C₁₂ to C₁₄ or C₁₃ to C₁₅ alkyl glycidyl ethers.

It is preferable when the epoxy resin composition contains at least one further constituent selected from the group consisting of diluents, accelerators, fillers, pigments, and surface-active additives.

Suitable diluents or accelerators especially include those mentioned hereinabove.

Suitable fillers are, in particular, ground or precipitated calcium carbonate, which is optionally coated with fatty acid, especially stearates, baryte (heavy spar), talc, quartz powder, quartz sand, silicon carbide, iron mica, dolomite, wollastonite, kaolin, mica (potassium aluminum silicate), molecular sieves, aluminum oxide, zinc oxide, aluminum-doped zinc oxide, aluminum hydroxide, magnesium hydroxide, silica, cement, gypsum, fly ash, carbon black, graphite, metal powders such as aluminum, copper, iron, zinc, silver or steel, PVC powder or hollow beads. Preference among these is given to calcium carbonate, baryte, quartz powder, talc, aluminum powder or a combination thereof.

Suitable pigments especially include titanium dioxides, iron oxides, chromium(III) oxides, organic pigments, carbon black or anticorrosion pigments, especially phosphates, orthophosphates or polyphosphates containing especially chromium, zinc, aluminum, calcium, strontium or a combination of these metals as counterions. Titanium dioxides are particularly suitable.

Suitable surface-active additives are especially defoamers, deaerating agents, wetting agents, dispersants, levelling agents and/or dispersed paraffin waxes.

The epoxy resin composition may optionally comprise further auxiliaries and additives, especially the following:
reactive diluents, especially those already mentioned, or epoxidized soybean oil or linseed oil, compounds containing acetoacetate groups, especially acetoacetylated polyols, butyrolactone, carbonates, aldehydes, isocyanates or silicones having reactive groups;
polymers, especially polyamides, polysulfides, polyvinyl formal (PVF), polyvinyl butyral (PVB), polyurethanes (PUR), polymers having carboxyl groups, polyamides, butadiene-acrylonitrile copolymers, styrene-acrylonitrile copolymers, butadiene-styrene copolymers, homo- or copolymers of unsaturated monomers, especially from the group comprising ethylene, propylene, butylene, isobutylene, isoprene, vinyl acetate or alkyl (meth)acrylates, especially chlorosulfonated polyethylenes or fluorine-containing polymers or sulfonamide-modified melamines;
fibers, especially glass fibers, carbon fibers, metal fibers, ceramic fibers or polymer fibers such as polyamide fibers or polyethylene fibers;
nanofillers, especially carbon nanotubes;
rheology modifiers, especially thickeners or antisettling agents;
adhesion improvers, especially organoalkoxysilanes;
flame-retardant substances, especially the aluminum hydroxide or magnesium hydroxide fillers already mentioned, antimony trioxide, antimony pentoxide, boric acid (B(OH)₃), zinc borate, zinc phosphate, melamine borate, melamine cyanurate, ammonium polyphosphate, melamine phosphate, melamine pyrophosphate, polybrominated diphenyl oxides or diphenyl ethers, phosphates such as, in particular, diphenyl cresyl phosphate, resorcinol bis(diphenyl phosphate), resorcinol diphosphate oligomer, tetraphenylresorcinol diphosphite, ethylenediamine diphosphate, bisphenol A bis(diphenyl phosphate), tris(chloroethyl) phosphate, tris(chloropropyl) phosphate, tris(dichloroisopropyl) phosphate, tris[3-bromo-2,2-bis(bromomethyl)propyl] phosphate, tetrabromobisphenol A, bis(2,3-dibromopropyl ether) of bisphenol A, brominated epoxy resins, ethylenebis(tetrabromophthalimide), ethylenebis(dibromonorbornanedicarboximide), 1,2-bis(tribromophenoxy)ethane, tris(2,3-dibromopropyl) isocyanurate, tribromophenol, hexabromocyclododecane, bis(hexachlorocyclopentadieno)cyclooctane or chloroparaffins; or
stabilizers against oxidation, heat, light or UV radiation or biocides.

The epoxy resin composition preferably has only a low content of diluents. It preferably contains less than 20% by weight, particularly preferably less than 10% by weight, in particular less than 5% by weight, most preferably less than 1% by weight, of diluent.

The epoxy resin composition may contain water.

In one embodiment the epoxy resin composition is water-based. The epoxy resin is preferably emulsified in an amount of 50% to 85% by weight in water and the hardener component preferably contains 20% to 80% by weight of water.

The epoxy resin composition preferably contains only a low content of water, preferably less than 5% by weight, especially less than 1% by weight, of water, however. Such a non-water-based epoxy resin composition is particularly versatile and particularly water-resistant.

Preference is given to an epoxy resin composition comprising
a resin component containing at least one epoxy resin and optionally further constituents such as in particular epoxy-containing reactive diluents, diluents, fillers, pigments and/or surface-active additives, and
a hardener component containing at least one amine of formula (I) and optionally further constituents such as in particular further amines, accelerators and/or diluents.

The resin component and the hardener component of the epoxy resin composition are stored in separate receptacles.

A suitable receptacle for storage of the resin component or the hardener component is in particular a vat, a hobbock, a bag, a bucket, a can, a cartridge or a tube. The components are storable, meaning that they can be stored prior to use for several months up to one year or longer without any change in their respective properties to a degree relevant to their use.

The resin component and hardener component are mixed shortly before or during application. The mixing ratio is preferably chosen such that the molar ratio of epoxy-reactive groups to epoxy groups is in the range from 0.5 to 1.5, especially 0.7 to 1.2. In parts by weight, the mixing ratio between the resin component and the hardener component is typically within a range from 1:2 to 20:1.

The components are mixed continuously or in batches by means of a suitable method, taking care to ensure that not too much time elapses between the mixing of the components and the application, and that application takes place within the pot life. Mixing and application can be effected at ambient temperature, which is typically in the range from about 5° C. to 40° C., preferably about 10° C. to 35° C., or at elevated temperature, especially in the range from 40° C. to 150° C., preferably 50° C. to 120° C.

Upon mixing the components, the curing of the epoxy resin composition by chemical reaction commences. Primary and secondary amino groups, and any further groups present that are reactive toward epoxy groups, react with the epoxy groups, resulting in ring opening thereof. As a result primarily of these reactions, the composition polymerizes and thereby cures.

Curing typically extends over a few hours to days. The duration depends on factors including the temperature, the reactivity of the constituents, the stoichiometry thereof, and the presence/amount of accelerators.

In the freshly mixed state the epoxy resin composition has a low viscosity. The viscosity 5 minutes after mixing of the resin component and the hardener component at 20° C. is preferably in the range from 0.1 to 20 Pa-s, preferably 0.2 to Pa-s, particularly preferably 0.3 to 5 Pa-s, measured using a cone-plate viscometer at a shear rate of 10 s⁻¹.

The epoxy resin composition is applied to at least one substrate and/or to at least one casting mold.

Suitable substrates are especially:
glass, glass ceramic, concrete, mortar, cement screed, fiber cement, brick, tile, plaster or natural rocks such as granite or marble,
repair compounds or levelling compounds based on PCC (polymer-modified cement mortar) or ECC (epoxy resin-modified cement mortar);
metals or alloys such as aluminum, iron, steel, copper, other nonferrous metals, including surface-finished metals or alloys such as galvanized or chrome-plated metals;
asphalt or bitumen;
leather, textiles, paper, wood, wood-based materials bonded with resins, for example phenolic, melamine or epoxy resins, resin-textile composites or further polymer composites;
plastics, such as rigid and flexible PVC, polycarbonate, polystyrene, polyester, polyamide, PMMA, ABS, SAN, epoxy resins, phenolic resins, PUR, POM, TPO, PE, PP, EPM or EPDM, in each case untreated or surface-treated, for example by means of plasma, corona or flames;
fiber-reinforced plastics, such as carbon fiber-reinforced plastics (CFRP), glass fiber-reinforced plastics (GFRP), and sheet molding compounds (SMC);
insulation foams, especially made of EPS, XPS, PUR, PIR, rock wool, glass wool or foamed glass;
coated or painted substrates, especially painted tiles, coated concrete, powder-coated metals or alloys or painted metal sheets;
coatings, paints or varnishes, especially coated floors that have been overcoated with a further floor covering layer.

The substrates can if required be pretreated prior to application, especially by physical and/or chemical cleaning methods or the application of an activator or a primer.

The substrates are especially coated and/or adhesively bonded.

A suitable casting mold is an apparatus into which the mixed, liquid epoxy resin composition can be poured and in which it can be cured, and from which it can be demolded or removed after curing, where the cured composition forms a shaped body.

The casting mold preferably consists at least of a material on the surface, from which the cured epoxy resin composition can be parted again without damage, especially made of metal, ceramic, plastic or silicone, optionally provided with a nonstick coating, especially of Teflon, silicone or a wax.

The invention further provides a cured composition obtained from the epoxy resin composition described after mixing of the resin component and the hardener component.

The epoxy resin composition is preferably used as a coating, primer, adhesive, sealant, potting compound, casting resin, impregnating resin, or as a shaped body or matrix for composite materials such as, in particular, CFRP (containing carbon fibers) or GFRP (containing glass fibers) or wood composites.

The use forms an article containing the cured composition composed of the described epoxy resin composition.

The article is in particular a floor coating, wall coating, component coating, pipe coating, roof coating or an anticorrosion coating or an adhesive-bonded article or a shaped body, in particular a composite material.

### EXAMPLES

The following are exemplary embodiments which are intended to more particularly elucidate the described invention. The invention is of course not limited to these described exemplary embodiments.
“AHEW” stands for amine hydrogen equivalent weight.

“EEW” stands for epoxy equivalent weight.

“Standard climatic conditions” (“SCC”) refer to a temperature of 23±1° C. and a relative atmospheric humidity of 50±5%.

The chemicals used were from Sigma-Aldrich Chemie GmbH, unless otherwise stated.

### Description of the Measurement Methods:

Viscosity was measured on a thermostated Rheotec RC30 cone-plate viscometer (cone diameter 50 mm, cone angle 1°, cone tip-plate distance 0.05 mm, shear rate s⁻¹). Viscosities of less than 100 mPa-s were measured with a shear rate of 100 s⁻¹.

Amine number was determined by titration (with 0.1 N HClO₄ in acetic acid against crystal violet).

Gas chromatograms (GC) were measured in the temperature range from 60° C. to 320° C. with a heating rate of 15° C./min and a 10 min hold time at 320° C. The injector temperature was 250° C. A Zebron ZB-5 column was used (L=30 m, ID=0.25 mm, dj=0.5 μm) at a gas flow of 1.5 ml/min. Detection was by flame ionization (FID).

Infrared spectra (FT-IR) were measured as undiluted films on a Nicolet iS5 FT-IR instrument from Thermo Scientific equipped with a horizontal ATR measurement unit with a diamond crystal. Absorption bands are reported in wavenumbers (cm⁻¹).

¹H NMR spectra were measured on a spectrometer of the Bruker Ascend 400 type at 400.14 MHz; the chemical shifts δ are reported in ppm relative to tetramethylsilane (TMS). No distinction was made between true coupling and pseudo-coupling patterns.

### Substances and Abbreviations Used

Araldite® GY 250: Bisphenol A diglycidyl ether, EEW approx. 187 g/eq (Huntsman)
Araldite® DY-E: Monoglycidyl ethers of C₁₂ to C₁₄ alcohols, EEW about 290 g/eq (Huntsman)
D.E.N.® 438: Phenol-formaldehyde novolac glycidyl ether, EEW approx. 179 g/eq, average functionality about 3.6 (Olin)
IPDA 3-aminomethyl-3,5,5-trimethylcyclohexylamine, AHEW 42.6 g/eq (Vestamin® IPD, Evonik)
MXDA: 1,3-bis(aminomethyl)benzene, AHEW 34 g/eq (Mitsubishi Gas Chemical)
Ancamine® K54 2,4,6-tris(dimethylaminomethyl)phenol (Evonik)

### Production of Amines:

Reaction Product P-1: (Containing N-furfuryl-1,2-ethanediamine; 1:1 Stoichiometry)

A round-bottomed flask was initially charged with 30.05 g (0.5 mol) of ethane-1,2-diamine under a nitrogen atmosphere at room temperature. With good stirring 48.05 g (0.5 mol) of furfural (furan-2-carbaldehyde, RCI=1) were added and stirred for a further 1 hour at 40° C. The reaction mixture was mixed with 1000 ml of isopropanol and then hydrogenated in a continuous hydrogenation apparatus with a Raney nickel fixed bed catalyst at a hydrogen pressure of 70 bar, a temperature of 70° C. and a flow rate of 5.5 ml/min. To monitor the reaction, IR spectroscopy was used to check whether the imine band at approx. 1665 cm⁻¹ had disappeared. The hydrogenated solution was then concentrated on a rotary evaporator at 65° C. to remove unreacted 1,2-ethanediamine, water and isopropanol. The reaction mixture thus obtained was a clear, slightly yellowish liquid having an amine number of 695 mg KOH/g, a viscosity at 20° C. of 10 mPa-s and a GC-determined content of N-furfuryl-1,2-ethanediamine of about 57.9% by weight (retention time 7.3 min), N-tetrahydrofurfuryl-1,2-ethanediamine of about 8.2% by weight (retention time 8.0 min), N,N′-difurfuryl-1,2-ethanediamine of about 31.6% by weight (retention time 11.9 min) and proportions of furan ring-hydrogenated N,N′-difurfuryl-1,2-ethanediamine of about 2.3% by weight (retention time 12.5 min). For further use, an AHEW of 58.9 g/eq was employed.

Reaction Product P-2: (Containing N-furfuryl-1,2-ethanediamine; 2:1 Stoichiometry)

A round-bottomed flask was initially charged with 60.1 g (1 mol) of ethane-1,2-diamine under a nitrogen atmosphere at room temperature. With good stirring 48.05 g (0.5 mol) of furfural (furan-2-carbaldehyde, RCI=1) were added and stirred for a further 1 hour at 40° C. The reaction mixture was mixed with 1000 ml of isopropanol and then hydrogenated in a continuous hydrogenation apparatus with a Raney nickel fixed bed catalyst at a hydrogen pressure of 70 bar, a temperature of 70° C. and a flow rate of 5.5 ml/min. To monitor the reaction, IR spectroscopy was used to check whether the imine band at approx. 1665 cm⁻¹ had disappeared. The hydrogenated solution was then concentrated on a rotary evaporator at 65° C. to remove unreacted 1,2-ethanediamine, water and isopropanol. The reaction mixture thus obtained was a clear, slightly yellowish liquid having an amine number of 772 mg KOH/g, a viscosity at 20° C. of 11 mPa-s and a GC-determined content of N-furfuryl-1,2-ethanediamine of about 78.2% by weight (retention time 7.3 min), N-tetrahydrofurfuryl-1,2-ethanediamine of about 12.3% by weight (retention time 8.0 min) and N,N′-difurfuryl-1,2-ethanediamine of about 9.1% by weight (retention time 11.9 min). For further use, an AHEW of 56.6 g/eq was employed.

Reaction Product P-3: (Containing N-furfuryl-1,2-ethanediamine; 3:1 Stoichiometry)

A round-bottomed flask was initially charged with 60.1 g (1 mol) of ethane-1,2-diamine under a nitrogen atmosphere at room temperature. With good stirring 32.0 g (0.33 mol) of furfural (furan-2-carbaldehyde, RCI=1) were added and stirred for a further 1 hour at 40° C. The reaction mixture was mixed with 1000 ml of isopropanol and then hydrogenated in a continuous hydrogenation apparatus with a Raney nickel fixed bed catalyst at a hydrogen pressure of 65 bar, a temperature of 65° C. and a flow rate of 5.5 ml/min. To monitor the reaction, IR spectroscopy was used to check whether the imine band at approx. 1665 cm⁻¹ had disappeared. The hydrogenated solution was then concentrated on a rotary evaporator at 65° C. to remove unreacted 1,2-ethanediamine, water and isopropanol. The reaction mixture thus obtained was a clear, slightly yellowish liquid having an amine number of 757 mg KOH/g, a viscosity at 20° C. of 10 mPa-s and a GC-determined content of N-furfuryl-1,2-ethanediamine of about 86.1% by weight (retention time 7.3 min), N-tetrahydrofurfuryl-1,2-ethanediamine of about 3.3% by weight (retention time 8.0 min), N,N′-difurfuryl-1,2-ethanediamine of about 2.8% by weight (retention time 11.9 min), proportions of furan ring-hydrogenated N,N′-difurfuryl-1,2-ethanediamine of about 1.5% by weight (retention time 12.5 min) and N,N,N′-trisfurfuryl-1,2-ethanediamine of about 6.0% by weight (retention time 14.2 min). For further use, an AHEW of 51.6 g/eq was employed.

Reaction Product P-4: (Containing N-furfuryl-1,2-ethanediamine; 3.5:1 Stoichiometry, Pd/C)

A round-bottomed flask was initially charged with 105.2 g (1.75 mol) of ethane-1,2-diamine under a nitrogen atmosphere at room temperature. With good stirring a solution of 48.05 g (0.5 mol) of furfural (furan-2-carbaldehyde, RCI=1) in 200 ml of isopropanol was added and stirred for a further 1 hour at 40° C. The reaction mixture was admixed with a further 1000 ml of isopropanol and then hydrogenated in a continuous hydrogenation apparatus with a Pd/C fixed bed catalyst at a hydrogen pressure of 80 bar, a temperature of 80° C. and a flow rate of 5 ml/min. To monitor the reaction, IR spectroscopy was used to check whether the imine band at approx. 1665 cm⁻¹ had disappeared. The hydrogenated solution was then concentrated on a rotary evaporator at 65° C. to remove unreacted 1,2-ethanediamine, water and isopropanol. The reaction mixture thus obtained was a clear, slightly yellowish liquid having an amine number of 691 mg KOH/g, a viscosity at 20° C. of 13.5 mPa-s and a GC-determined content of N-furfuryl-1,2-ethanediamine of about 72.8% by weight (retention time 7.3 min), N-tetrahydrofurfuryl-1,2-ethanediamine of about 7.8% by weight (retention time 8.0 min), N,N′-difurfuryl-1,2-ethanediamine of about 3.3% by weight (retention time 11.9 min), proportions of furan ring-hydrogenated N,N′-difurfuryl-1,2-ethanediamine of about 3.0% by weight (retention time 12.5 min), N,N,N′-trisfurfuryl-1,2-ethanediamine of about 9.9% by weight (retention time 14.2 min) and proportions of furan ring-hydrogenated N,N,N′-trisfurfuryl-1,2-ethanediamine of about 3.2% by weight (retention time about 14.7 min). For further use, an AHEW of 55 g/eq was employed.

### N-Furfuryl-1,2-ethanediamine (F-EDA)

41.2 g of the reaction product P-4 produced as described above were distilled under reduced pressure at 70° C., resulting in 25.6 g of distillate being collected at a vapor temperature of about 50° C. and 0.1 bar. This afforded a colorless liquid having an amine number of 802 mg KOH/g, an AHEW of about 46.7 g/eq, an RCI of 0.71, a viscosity of 3.2 mPa-s at 20° C. and a GC-determined content of N-furfuryl-1,2-ethanediamine of 94.6% by weight (retention time 7.3 min) and N-tetrahydrofurfuryl-1,2-ethanediamine of 5.3% by weight (retention time 8.0 min) which was hereinbelow employed as F-EDA.

¹H NMR (CDCl₃): 7.33 (d, 1H, Ar—H), 6.27 (m, 1H, Ar—H), 6.14 (m, 1H, Ar—H), 3.76 (s, 2H, Ar—CH₂), 2.78 (m, 2H, NHCH₂CH₂), 2.65 (m, 2H, CH₂NH₂), 1.52 (br s, 3H, NH and NH₂).

FT-IR: 3284, 3043, 2945, 2838, 1567, 1504, 1455, 1382, 1306, 1219, 1146, 1108, 1073, 1009, 916, 883, 806, 738.

### N,N′-Difurfuryl-1,2-ethanediamine (BisF-EDA)

A round-bottomed flask was initially charged with 11.12 g (0.185 mol) of 1,2-ethanediamine in 200 ml of isopropanol under a nitrogen atmosphere at room temperature. With good stirring 35.0 g (0.37 mol) of furfural (furan-2-carbaldehyde, RCI=1) were added and the mixture was stirred for a further 1 hour at 40° C. The reaction mixture was admixed with a further 800 ml of isopropanol and then hydrogenated in a continuous hydrogenation apparatus with a Raney nickel fixed bed catalyst at a hydrogen pressure of 65 bar, a temperature of 65° C. and a flow rate of 5.5 ml/min. To monitor the reaction, IR spectroscopy was used to check whether the imine band at approx. 1665 cm⁻¹ had disappeared. The hydrogenated solution was then concentrated on a rotary evaporator at 65° C. to remove unreacted 1,2-ethanediamine, water and isopropanol. This afforded a clear, slightly yellowish liquid which was distilled under vacuum at 110° C. to 130° C., the distillate being collected at a vapor temperature of 105° C. to 110° C. and 0.15 bar. This afforded a colorless liquid having an amine number of 502 mg KOH/g, an AHEW of about 110 g/eq, a viscosity of 28 mPa-s at 20° C. and a GC-determined content of N,N′-difurfuryl-1,2-ethanediamine of 79.0% by weight (retention time 11.9 min), proportions of furan ring-hydrogenated N,N′-difurfuryl-1.2-ethanediamine of about 11.9% by weight (retention time 12.4 to 12.5 min), N,N,N′-trisfurfuryl-1,2-ethanediamine of about 4.4% by weight (retention time 14.2 min) and proportions of furan ring-hydrogenated N,N,N′-trisfurfuryl-1,2-ethanediamine of about 3.7% by weight (retention time about 14.6 to 14.7 min) which was hereinbelow employed as BisF-EDA.

### N-Tetrahydrofurfuryl-1,2-ethanediamine (THF-EDA)

A round-bottomed flask was initially charged with 60.1 g (1 mol) of ethane-1,2-diamine under a nitrogen atmosphere at room temperature. A solution of 32.0 g (0.33 mol) of furfural in 200 ml of isopropanol was slowly added dropwise with good stirring and stirring was continued at 40° C. for a further 1 hour. The reaction mixture was admixed with a further 300 ml of isopropanol and then hydrogenated in a continuous hydrogenation apparatus with a Raney nickel fixed bed catalyst at a hydrogen pressure of 90 bar, a temperature of 110° C. and a flow rate of 5 ml/min. To monitor the reaction, IR spectroscopy was used to check whether the imine band at approx. 1665 cm⁻¹ had disappeared. The hydrogenated solution was then concentrated on a rotary evaporator at 65° C. to remove unreacted 1,2-ethanediamine, water and isopropanol.

This afforded a clear, slightly yellowish liquid which was distilled under vacuum at 70° C., 35.6 g of distillate being collected at a vapor temperature of about 50° C. and 0.1 bar. This afforded a colorless liquid having a viscosity of 4.3 mPa-s at 20° C., an amine number of 728 mg KOH/g, an AHEW of 48.1 g/eq and a GC-determined content of N-tetrahydrofurfuryl-1,2-ethanediamine of 97% by weight (retention time 8.0 min) which was hereinbelow employed as THF-EDA.

### N-Benzyl-1,2-ethanediamine (B-EDA)

180.3 g (3 mol) of 1,2-ethanediamine were initially charged at room temperature, mixed with a solution of 106.0 g (1 mol) of benzaldehyde in 1200 ml of isopropanol, stirred for 2 hours and then hydrogenated at 80° C., 80 bar of hydrogen pressure and a flow rate of 5 ml/min in a continuous hydrogenation apparatus with a Pd/C fixed bed catalyst and the hydrogenated solution was concentrated on a rotary evaporator at 65° C. to remove unreacted 1,2-ethanediamine, water and isopropanol. The reaction mixture thus obtained was a clear, pale yellowish liquid having a GC-determined content of N-benzyl-1,2-ethanediamine of about 81% by weight (retention time 8.5 min) and of N,N′-dibenzylethane-1,2-diamine of about 14% by weight (retention time 14.3 min). Said mixture was purified by distillation at 80° C. under reduced pressure. This afforded a colorless liquid having an AHEW of 50.1 g/eq and a GC-determined content of N-benzyl-1,2-ethanediamine of >97% which was hereinbelow employed as B-EDA.

### Preparation of Adducts:

### Adduct A1:

51.3 g of N-furfuryl-1,2-ethanediamine (F-EDA, 0.366 mol) were heated to 70° C. and with good stirring 45.0 g of Araldite® GY 250 (0.241 mol of EP groups) were slowly added, wherein the temperature of the reaction mixture was maintained between 70° C. and 90° C. The reaction mixture was stirred at this temperature range for one hour and subsequently cooled. This afforded a clear, slightly yellowish liquid having a viscosity at 20° C. of 124 Pa-s, an amine number of 419 mg KOH/g and a calculated AHEW of 112.3 g/eq.

### Adduct A2:

58.6 g of N-furfuryl-1,2-ethanediamine (F-EDA, 0.418 mol) were heated to 70° C. and with good stirring 37.3 g of D.E.N.® 438 (0.208 mol of EP groups) were slowly added, wherein the temperature of the reaction mixture was maintained between 70° C. and 90° C. The reaction mixture was stirred at this temperature range for one hour and subsequently cooled. This afforded a clear, slightly yellowish liquid having a viscosity at 20° C. of 40.8 Pa-s, an amine number of 492 mg KOH/g and a calculated AHEW of 91.6 g/eq.

### Adduct A3 (Ref.):

55.0 g of N-benzylethane-1,2-diamine (B-EDA, 0.366 mol) was heated to 70° C. and with good stirring 45.0 g of Araldite® GY 250 (0.241 mol of EP groups) were slowly added, wherein the temperature of the reaction mixture was maintained between 70° C. and 90° C. The reaction mixture was kept within this temperature range for one hour and then cooled. This afforded a clear, slightly yellowish liquid having a viscosity at 20° C. of 262 Pa-s, an amine number of 408 mg KOH/g and a calculated AHEW of 116.3 g/eq.

### Production of Epoxy Resin Compositions:

### Examples Z-1 and Ref-1 to Ref-3

For each example, the resin component and hardener component specified in table 1 were heated separately to a temperature of 60° C. These preheated components were then used to produce a portion of altogether 20 g of epoxy resin composition by mixing the components in the weight ratio specified in table 1 using a centrifugal mixer (SpeedMixer™ DAC 150, FlackTek Inc.) for 15 seconds and then immediately performing testing as follows:

The mixed composition was introduced into a test tube thermostatted to 60° C. using a water bath and a temperature sensor was positioned in the middle of the mixed material. This was used to determine the time until attainment of the maximum temperature (reported in the table as time to peak exotherm) and the maximum temperature level (peak exotherm temperature) in the mixed material. The values given in table 1 are average values from three measurements.

The Tg value (glass transition temperature) was measured by DSC on cured samples from the middle of the test tube from the above-described determination, and these samples were additionally stored under standard climatic conditions before the measurement for 14 days. The measurement was effected with a Mettler Toledo DSC 3+ 700 instrument and the measurement program (1) −10° C. for 2 min, (2) -10 to 200° C. at a heating rate of 10 K/min (=1st run), (3) 200 to −10° C. at a cooling rate of −50 K/min, (4) −10° C. for 2 min, (5) −10 to 180° C. at a heating rate of 10 K/min (=2nd run).

The results are reported in table 1.

The epoxy resin composition Z-1 is an inventive example. The epoxy resin compositions Ref-1 to Ref-3 are comparative examples.

TABLE 1
Composition and properties of Z-1 and Ref-1 to Ref-2.

| Example | Z-1 | Ref-1 | Ref-2 | Ref-3 |
|---|---|---|---|---|
| Resin comp.: | | | | |
| Araldite ® GY 250 | 187.0 | 187.0 | 187.0 | 187.0 |
| Hardener comp.: | | | | |
| F-EDA | 46.7 | — | — | — |
| B-EDA | — | 50.1 | — | — |
| MXDA | — | — | 34.0 | — |
| IPDA | — | — | — | 42.6 |
| time to peak exotherm [min] | 14 | 12 | 12 | 21 |
| peak exotherm temperature | 106° C. | 201° C. | 150° C. | 74° C. |
| Tg 1st/2nd run [° C.] | 50/90 | 53/88 | 93/100 | 76/155 |

### Examples Z-2 to Z-11 and Ref-4 to Ref-8

For each example, the ingredients of the resin component reported in tables 2 to 4 were mixed in the specified amounts (in parts by weight) using a centrifugal mixer (SpeedMixer™ DAC 150, FlackTek Inc.) and stored with exclusion of moisture. The ingredients of the hardener component specified in tables 2 to 4 were also processed and stored.

The two components of each composition were then processed to afford a homogeneous liquid using the centrifugal mixer and said liquid was immediately tested as follows:

Viscosity was measured as described at a temperature of 20° C. 5 min after mixing the resin component and the hardener component.

Gel time was determined by moving a freshly mixed amount of about 3 g under standard climatic conditions with a spatula at regular intervals until the mass underwent gelation.

Shore D hardness was determined in accordance with DIN 53505 on two cylindrical test specimens (diameter 20 mm, thickness 5 mm), one of which was stored under standard climatic conditions and the other at 8° C. and 80% relative humidity, and the hardness measured in each case after 1 day (24 h) and after 2 days.

In addition, a film was applied to a glass plate in a layer thickness of 500 m, and this was stored/cured under standard climatic conditions. König's hardness (König's pendulum hardness to DIN EN ISO 1522) was determined on this film after 1 day, 2 days, 7 days and 14 days (1d SCC), (2d SCC), (7d SCC), (14d SCC). After 14 days, the appearance (SCC) of the film was assessed. A clear film was described as “attractive” if it had a glossy and nontacky surface with no structure. “Structure” refers to any kind of marking or pattern on the surface. A further film was applied to a glass plate in a layer thickness of 500 m and, immediately after application, stored/cured at 8° C. and 80% relative humidity for 7 days and then under standard climatic conditions for 2 weeks. 24 hours after application, a polypropylene bottle top beneath which a damp sponge had been positioned was placed on the film. After a further 24 hours, the sponge and the bottle top were removed and positioned at a new point on the film, from which they were in turn removed and repositioned after 24 hours, this being done a total of 4 times. The appearance of this film was then assessed (referred to as “appearance (8°/80%)” in the tables) in the same way as described for appearance (SCC). Also reported in each case here was the number and nature of visible marks that had formed in the film as a result of the damp sponge or the bottle top on top. The number of white discolored spots was referred to as “blushing”. A faint white discolored spot was designated as “(1)”. A clear white discolored spot was designated as “1”. The designation “ring” was reported if a ring-shaped imprint was present due to sinking-in of the first bottle top applied 24 hours after application. Such a ring-shaped impression indicates that the coating was not ready to be walked on. The films cured in this way in turn had their König hardness determined, in each case after 7 days at 8° C. and 80% relative humidity (König (7d 8°/80%)), then after a further 2 days under SCC (König (+2d SCC)) or 7 days under SCC (König (+7d SCC)) or 14 days under SCC (König (+14d SCC)).

The results are shown in tables 2 to 4.

The epoxy resin compositions Z-2 to Z-10 are inventive examples. The epoxy resin compositions Ref-4 to Ref-8 are comparative examples.

TABLE 2
Composition and properties of Z-2 to Z-4 and Ref-4 to Ref-6.

| Example | Z-2 | Ref-4 | Ref-5 | Z-3 | Z-4 | Ref-6 |
|---|---|---|---|---|---|---|
| Resin component: | | | | | | |
| Araldite ® GY-250 | 167.2 | 167.2 | 167.2 | 167.2 | 167.2 | 167.2 |
| Araldite ® DY-E | 31.8 | 31.8 | 31.8 | 31.8 | 31.8 | 31.8 |
| Hardener component: | | | | | | |
| F-EDA | 46.7 | — | — | — | — | — |
| THF-EDA | — | 48.1 | — | — | — | — |
| B-EDA | — | — | 50.1 | — | — | — |
| Adduct A1 | — | — | — | 112.3 | — | — |
| Adduct A2 | — | — | — | — | 91.6 | — |
| Adduct A3 | — | — | — | — | — | 116.3 |
| Viscosity (10′) [Pa · s] | 0.20 | 0.20 | 0.22 | 13.8 | 5.3 | 14.6 |
| Gel time (h:min) | 5:00 | >5:30 | 5:15 | 2:30 | 2:30 | 2:30 |
| Shore D (1 d SCC) | 75 | 67 | 75 | 74 | 80 | 73 |
| (2 d SCC) | 78 | 73 | 76 | 76 | 82 | 75 |
| Shore D (1 d 8°/80%) | n.m.¹ | n.m.¹ | n.m.¹ | 61 | 56 | 63 |
| (2 d 8°/80%) | 61 | 49 | 62 | 74 | 70 | 76 |
| König hardness(1 d SCC) | 41 | 24 | 116 | 162 | 137 | 175 |
| [s] (2 d SCC) | 63 | 43 | 148 | 192 | 182 | 204 |
| (7 d SCC) | 119 | 91 | 190 | 203 | 203 | |
| (14 d SCC) | 167 | 111 | 197 | | | |
| Appearance (SCC) | attractive | attractive | attractive | attractive | attractive | attractive |
| König (7 d 8°/80%) | 12 | n.m.¹ | 29 | 50 | 35 | 109 |
| [s] (+2 d SCC) | 25 | n.m.¹ | 67 | 102 | 59 | 190 |
| (+7 d SCC) | 52 | n.m.¹ | 90 | 164 | 83 | 195 |
| (+14 d SCC) | 61 | 6 | 104 | 171 | 124 | 197 |
| Appearance (8°/80%) | attractive | Structure | attractive | attractive | attractive | attractive |
| Blushing | 1 | 4 | (1) | (1) | (1) | none |
| Ring | 1 | 2 | none | none | none | none |

¹not measurable (too soft)

TABLE 3
Composition and properties of Z-5 to Z-6 and Ref-7 to Ref-8.

| Example | | Z-5 | Ref-7 | Z-6 | Ref-8 |
|---|---|---|---|---|---|
| Resin component: | | | | | |
| Araldite ® GY-250 | | 167.2 | 167.2 | 167.2 | 167.2 |
| Araldite ® DY-E | | 31.8 | 31.8 | 31.8 | 31.8 |
| Hardener component: | | | | | |
| F-EDA | | 33.1 | — | 14.5 | — |
| B-EDA | | — | 35.1 | — | 15.0 |
| Adduct A1 | | 33.1 | — | 33.1 | — |
| Adduct A3 | | — | 35.1 | — | 35.1 |
| IPDA | | — | — | 17.0 | 17.0 |
| Benzyl alcohol | | — | — | 20.0 | 20.0 |
| Ancamine ® K54 | | — | — | 2.0 | 2.0 |
| Viscosity (10′) [Pa · s] | | 0.66 | 0.75 | 0.82 | 0.92 |
| Gel time (h:min) | | 3:40 | 3:40 | 3:10 | 3:20 |
| Shore D | (1 d SCC) | 79 | 78 | 75 | 77 |
| | (2 d SCC) | 80 | 79 | 77 | 78 |
| Shore D | (1 d 8°/80%) | 23 | 38 | 31 | 38 |
| | (2 d 8°/80%) | 58 | 66 | 67 | 71 |
| König | (1 d SCC) | 94 | 136 | 55 | 66 |
| hardness | (2 d SCC) | 133 | 181 | 101 | 108 |
| [s] | (7 d SCC) | 153 | 198 | 136 | 155 |
| | (14 d SCC) | 199 | 209 | 167 | 168 |
| Appearance (SCC) | | attractive | attractive | attractive | attractive |
| König | (7 d 8°/80%) | 15 | 45 | 21 | 29 |
| [s] | (+2 d SCC) | 46 | 130 | 95 | 119 |
| | (+7 d SCC) | 64 | 146 | 119 | 153 |
| | (+14 d SCC) | 83 | 152 | 129 | 155 |

| | | | | |
|---|---|---|---|---|
| Appearance (8°/80%) | attractive | attractive | attractive | attractive |
| Blushing | (1) | (1) | (1) | (1) |
| Ring | none | none | none | none |

TABLE 4
Composition and properties of Z-7 to Z-11.

| Example | | Z-7 | Z-8 | Z-9 | Z-10 | Z-11 |
|---|---|---|---|---|---|---|
| Resin component: | | | | | | |
| Araldite ® GY-250 | | 167.2 | 167.2 | 167.2 | 167.2 | 167.2 |
| Araldite ® DY-E | | 31.8 | 31.8 | 31.8 | 31.8 | 31.8 |
| Hardener component: | | | | | | |
| Reaction product P-1 | | 58.9 | — | — | — | — |
| Reaction product P-2 | | — | 56.6 | — | — | — |
| Reaction product P-3 | | — | — | 51.6 | — | — |
| Reaction product P-4 | | — | — | — | 55.0 | — |
| BisF-EDA | | — | — | — | — | 110.0 |
| Viscosity (10′) [Pa · s] | | 0.22 | 0.18 | 0.23 | 0.28 | 0.17 |
| Gel time (h:min) | | 6:25 | 5:05 | 5:10 | >7:00 | >7:00 |
| Shore D | (1 d SCC) | 67 | 74 | 74 | 40 | n.m.¹ |
| | (2 d SCC) | 74 | 77 | 79 | 50 | n.m.¹ |
| Shore D | (1 d 8°/80%) | n.m.¹ | n.m.¹ | n.m.¹ | n.m.¹ | n.m.¹ |
| | (2 d 8°/80%) | 59 | 70 | 67 | 48 | n.m.¹ |
| Konig | (1 d SCC) | 31 | 49 | 29 | 8 | n.m.² |
| hardness | (2 d SCC) | 60 | 56 | 39 | 15 | 3 |
| [s] | (7 d SCC) | 81 | 96 | 77 | 84 | 8 |
| | (14 d SCC) | 113 | 118 | 102 | 96 | 8 |

| | | | | | |
|---|---|---|---|---|---|
| Appearance (SCC) | attractive | attractive | attractive | attractive | attractive |

¹not measurable (too soft)
²not measurable (tacky)

1. The use of a hardener containing at least one amine of formula (I) for curing of epoxy resins, 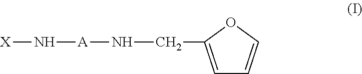
wherein A represents a linear alkylene radical having 2 to 10 carbon atoms and X represents H or furfuryl.

2. The use as claimed in claim 1, characterized in that A represents a radical selected from the group consisting of 1,2-ethylene, 1,3-propylene, 1,4-butylene, 1,5-pentylene and 1,6-hexylene.

3. The use as claimed in claim 1, characterized in that A represents 1,2-ethylene.

4. The use as claimed in claim 1, characterized in that X represents H.

5. The use as claimed in claim 1, characterized in that the amine of formula (I) has an RCI of at least 0.45, preferably at least 0.6, in particular at least 0.7, most preferably of 1, wherein the RCI is the ratio of the number of carbon atoms from biobased sources to the total number of carbon atoms of the amine of formula (I).

6. The use as claimed in claim 1, characterized in that the amine of formula (I) is employed in the form of a reaction product obtained from the reductive alkylation of at least one amine of formula H₂N-A-NH₂ with furfural and hydrogen and subsequent removal of unreacted amine of formula H₂N-A-NH₂.

7. The use as claimed in claim 6, characterized in that the molar ratio of the amine of formula H₂N-A-NH₂ to furfural is in the range from 1 to 2, preferably 1 to 1.5.

8. The use as claimed in claim 1, characterized in that X represents H and the hardener additionally contains an amine of formula 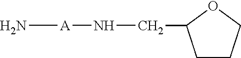
in a weight ratio between the amine of formula (I) and the amine of formula 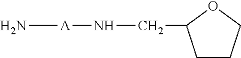
in the range from 70/30 to 99/1, preferably 80/20 to 98/2.

9. The use as claimed in claim 1, characterized in that the amine of formula (I) is present partially or completely in the form of an amine-functional adduct with at least one epoxy resin or monoepoxide in a stoichiometric ratio of at least 1 mol of amine of formula (I) to 1 mol equivalent of epoxy groups.

10. The use as claimed in claim 1, characterized in that the hardener contains at least one further constituent selected from further amines which do not conform to formula (I), accelerators and diluents, in particular at least one further amine which does not conform to formula (I).

11. The use as claimed in claim 10, characterized in that the hardener contains as a further amine which does not conform to formula (I) at least one amine having an RCI of 1, in particular selected from 2,5-bis(aminomethyl)furan, 2,5-bis(aminomethyl)tetrahydrofuran, bis(5-aminomethylfuran-2-yl)methane, bis(5-aminomethyltetrahydrofuran-2-yl)methane, 2,2-bis(5-aminomethylfuran-2-yl)propane and 2,2-bis(5-aminomethyltetrahydrofuran-2-yl)propane, wherein the RCI is the ratio of the number of carbon atoms from biobased sources to the total number of carbon atoms of the amine.

12. An epoxy resin composition comprising
a resin component comprising at least one epoxy resin and
a hardener component comprising the hardener as described in claim 1.

13. A cured epoxy resin composition obtained from the epoxy resin composition as claimed in claim 12 after the mixing of the resin component and the hardener component.

14. A reaction product obtained from the reductive alkylation of an amine of formula H₂N-A-NH₂ with furfural and hydrogen in a molar ratio of the amine of formula H₂N-A-NH₂ to furfural in the range from 1 to 2, and subsequent removal of amine of formula H₂N-A-NH₂ to a content of at most 1% by weight, based on the reaction product, wherein A represents a linear alkylene radical having 2 to 10 carbon atoms.

15. The reaction product as claimed in claim 14, wherein A represents 1,2-ethylene and the reaction product contains
50% to 80% by weight of N-furfuryl-1,2-ethanediamine,
5% to 50% by weight of N,N′-difurfuryl-1,2-ethanediamine,
0% to 20% by weight of N-tetrahydrofurfuryl-1,2-ethanediamine,
less than 1% weight of 1,2-ethanediamine
and optionally further constituents based on the reaction product.

16. An amine mixture containing at least one amine of formula 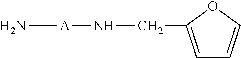
and at least one amine of formula 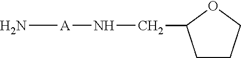
in a weight ratio in the range from 70/30 to 99/1, wherein A represents a linear alkylene radical having 2 to 10 carbon atoms.
